(19) United States
(12) Patent Application Publication
(10) Pub. No.: US 2018/0325447 A1
(43) Pub. Date: Nov. 15, 2018

(54) MULTI-TESTING MEDICAL/AMBIOFEEDBACK UNIT WITH GRAPHIC INTERFACE

(71) Applicants: Paul Hriso, Bedminster, NJ (US); Richard van Riet, Bayonne, NJ (US)

(72) Inventors: Paul Hriso, Bedminster, NJ (US); Richard van Riet, Bayonne, NJ (US)

(21) Appl. No.: 15/972,777

(22) Filed: May 7, 2018

Related U.S. Application Data
(60) Provisional application No. 62/501,814, filed on May 5, 2017.

(51) Int. Cl. A61B 5/00; A61B 5/0205; G16H 40/67

(52) U.S. Cl. CPC A61B 5/486; A61B 5/0205; A61B 5/0002; A61B 5/02416; A61B 5/7475; G16H 40/67; A61B 5/0077; A61B 5/7435

(57) ABSTRACT

Systems and methods for providing multiple medical tests to a user are provided. The system includes a computing device, the computing device, including a memory configured to store user data one or more clinical interventions and a processor configured to run the one or more clinical interventions. The system further includes one or more sensors coupled to the computing device, each of the one or more sensors configured to capture data from the user and send the data to the computing device, one or more displays coupled to the computing device, the one or more displays configured to display the one or more clinical interventions, and a remote server coupled to the computing device.

### CLAIM OF PRIORITY

This application claims priority to U.S. Provisional Patent Application No. 62/501,814, filed May 5, 2017, incorporated by reference herein in its entirety.

### FIELD OF THE EMBODIMENTS

This invention relates to medical sensing and testing systems and methods and, in particular, to multi-testing medical sensing and testing systems and methods the incorporate photorealistic imagery.

### BACKGROUND OF THE EMBODIMENTS

Biofeedback is a method of treating various ailments and conditions a patient may have. In traditional biofeedback, the biofeedback unit is primarily a sensor signal translator and the clinician puts out positive interventions (relaxation, guided imageries, breathing techniques, etc.) or negative interventions (stress inducers, etc.) and measures the responses from the patients. However, according to this type of treatment, a simple session treatment timeline is created through which a patient is to follow a set of interventions. These interventions are not dynamically altered based on results gathered from current results. Thus dynamic biofeedback system is needed to more efficiently administer treatment to patients.

Examples of related art are described below:

U.S. Pat. No. 9,532,748 generally describes technologies and solutions that address global acquisition and storage of acquired EEG data and processed EEG data, development interfaces for expansion and re-analysis of acquired EEG data, integration to other non-EEG derived user data, and long-term user wearability.

U.S. Patent Publication No. 2003/0130566 generally describes a method for influencing mental or physical states of an individual, using visual imagery, wherein the visual imagery is created using source image components derived from physical structures or materials that are causally associated with, the mental or physical state that is sought to be influenced, or which is desired to be created. The present invention is also directed to the visual images that are created, as well as to articles bearing the visual images that have been created, for purposes of exercising the method.

U.S. Patent Publication No. 2014/0316192 generally describes biofeedback virtual reality sleep assistant technologies that monitor one or more physiological parameters while presenting an immersive environment. The presentation of the immersive environment changes over time in response to changes in the values of the physiological parameters. The changes in the presentation of the immersive environment are configured using biofeedback technology and are designed to promote sleep.

U.S. Patent Publication No. 2016/0317041 generally describes a system and methods for measuring physiological parameters. More specifically, a noncontact technology by which one or more physiological parameters of a subject may be efficiently and quickly detected. Among other advantages, the present invention can be used to assess and monitor vital signs of one or more subjects in a variety of contexts including for medical or security triage purposes, for use in healthcare waiting rooms, as part of human imaging systems, or during surgery.

U.S. Patent Publication No. 2016/0360970 generally describes wearable devices for taking symmetric thermal measurements. One device includes first and second thermal cameras physically coupled to a frame worn on a user's head. The first thermal camera takes thermal measurements of a first region of interest that covers at least a portion of the right side of the user's forehead. The second thermal camera takes thermal measurements of a second ROI that covers at least a portion of the left side of the user's forehead. Wherein the first and second thermal cameras are not in physical contact with their corresponding ROIs, and as a result of being coupled to the frame, the thermal cameras remain pointed at their corresponding ROIs when the user's head makes angular movements.

U.S. Patent Publication No. 2016/0366462 generally describes methods and systems for a media guidance application configured to generate for display an icon that provides feedback to a user related to the current brain activity of the user. For example, a media guidance application may monitor the brain activity of the user in order to determine whether or not to perform a particular operation. The media guidance application may further generate a display of icons that inform the user of the current brain activity of the user and/or the progress of the user towards achieving a particular operation.

U.S. Patent Publication No. 2016/0371721 generally describes methods and systems for determining whether a viewer watched an advertisement. In some embodiments, control circuitry generates for display an advertisement including a stimulus. For example, the stimulus may be incorporated into the advertisement by a content provider in order to trigger a region of the viewer's brain. The control circuitry monitors brain activity in the region of the viewer's brain during the advertisement. The control circuitry determines a brain state associated with the region based on the brain activity of the viewer during the advertisement. The control circuitry compares the brain state to a threshold range associated with the stimulus included in the advertisement. In response to determining that the brain state matches the threshold range, the control circuitry stores in a database an indication that the viewer watched the advertisement.

International Patent Publication No. WO2015175838 generally describes sensing devices including pliable e-textile pressure sensors are used in gloves intended to be worn against a user's hand, or a portion of a user's hand. Additional sensors, such as accelerometer(s), gyroscope(s) and geo-referencing sensor(s), may be incorporated in electronic devices that interface electronically with the pressure sensors and are mounted on or in proximity to a glove when in use. Systems and methods for storing, communicating, processing, analyzing and displaying data collected by sensor components for remote monitoring of conditions at hand surfaces, position and orientation data, movement data, and the like, are also disclosed. Sensors and sensor systems provide substantially real-time feedback relating to current body conditions, orientation and movement, and may provide notifications or alerts to users, coaches, etc., enabling early intervention when conditions indicate intervention is appropriate.

International Patent Publication No. WO2016110804A1 generally describes one or more wearable devices (i.e. attached or applied to limbs, body, head or other body extremities but also applicable to implanted or physiologically attachable systems). These systems have a means of enabling diagnostic or prognostic monitoring applicable to monitoring relevant parameters and corresponding analysis determination and characterization applicable to the onset or detection of events or health conditions of interest. One application relates to sleep monitoring and associate EEG sensors.

None of the art described above addresses all of the issues that the present invention does.

Various systems and methodologies are known in the art. However, their structure and means of operation are substantially different from the present disclosure. The other inventions fail to solve all the problems taught by the present disclosure. At least one embodiment of this invention is presented in the drawings below and will be described in more detail herein.

### SUMMARY OF THE EMBODIMENTS

According to an aspect of the present invention, a system is provided for providing multiple medical tests to a user. The system includes a computing device, the computing device including a memory configured to store user data one or more clinical interventions and a processor configured to run the one or more clinical interventions. The system further includes one or more sensors coupled to the computing device, each of the one or more sensors configured to capture data from the user and send the data to the computing device, one or more displays coupled to the computing device, the one or more displays configured to display the one or more clinical interventions, and a remote server coupled to the computing device.

According to another aspect of the present invention, a method for providing multiple medical tests to a user is provided. The method includes programming, on a computing device, a series of clinical interventions to be performed by a user, the computing device including a memory configured to store user data one or more clinical interventions, and a processor configured to run the one or more clinical interventions. The method further includes coupling one or more sensors to the user, wherein the one or more sensors are coupled to the computing device, displaying to the user, on one or more displays, one or more clinical interventions from the series of clinical interventions, capturing data from the user, using the one or more sensors, sending the data to the computing device, and analyzing the data, using the computing device.

It is an object of the present invention to provide the system for providing multiple medical tests to the user, wherein at least one of the one or more sensors are wirelessly coupled to the computing device.

It is an object of the present invention to provide the system for providing multiple medical tests to the user, wherein the computing device further includes, one or more adapters configured to receive one or more inputs from the one or more sensors.

It is an object of the present invention to provide the system for providing multiple medical tests to the user, wherein the one or more sensors are selected from the group consisting of a PulseOx, an ECG, a spirometry sensor, a skin conductance sensor, an infrared camera, an EMG, an EEG, a facial expression recognition sensor, a capnometer, a pneumograph, a photoplethismogram, a rheoencephalogram, a hemoencephalogram, an ultrasound sensor, and an ultrasound transducer.

It is an object of the present invention to provide the system for providing multiple medical tests to the user, wherein the computing device is configured to cause the display to project a photorealistic display of an environment to coincide with one or more clinical interventions.

It is an object of the present invention to provide the system for providing multiple medical tests to the user, wherein the computing device is configured to analyze data captured by the one or more sensors in order to determine one or more possible alterations to a regimen of clinical interventions in a timeline of interventions.

It is an object of the present invention to provide the system for providing multiple medical tests to the user, wherein the computing device is configured to display, to a second user, the one or more possible alterations.

It is an object of the present invention to provide the system for providing multiple medical tests to the user, further comprising a graphical user interface coupled to the computing device.

It is an object of the present invention to provide the system for providing multiple medical tests to the user, wherein the remote server is coupled to one or more secondary computing devices.

It is an object of the present invention to provide the system for providing multiple medical tests to the user, wherein the one or more displays are selected from the group consisting of a 2-dimensional television screen, a 3-dimensional television screen, a 2-dimensional computer display, a 3-dimensional computer display, a projector, a virtual reality apparatus, and an augmented reality apparatus.

It is an object of the present invention to provide the system for providing multiple medical tests to the user, wherein the one or more sensors are incorporated into the computing device.

It is an object of the present invention to provide the method for providing multiple medical tests to the user, wherein at least one of the one or more sensors are wirelessly coupled to the computing device.

It is an object of the present invention to provide the method for providing multiple medical tests to the user, wherein the computing device further includes one or more adapters configured to receive one or more inputs from the one or more sensors.

It is an object of the present invention to provide the method for providing multiple medical tests to the user, wherein the one or more sensors are selected from the group consisting of a PulseOx, an ECG, a spirometry sensor, a skin conductance sensor, an infrared camera, an EMG, an EEG, a facial expression recognition sensor, a capnometer, a pneumograph, a photoplethismogram, a rheoencephalogram, a hemoencephalogram, an ultrasound sensor, and an ultrasound transducer.

It is an object of the present invention to provide the method for providing multiple medical tests to the user, further comprising sending a signal from the computing device to the one or more displays to cause the one or more displays to project a photorealistic display of an environment to coincide with one or more clinical interventions.

It is an object of the present invention to provide the method for providing multiple medical tests to the user, wherein the analyzing the data further includes determining one or more possible alterations to series of clinical interventions based on the data.

It is an object of the present invention to provide the method for providing multiple medical tests to the user, further comprising displaying, to a second user, the one or more possible alterations.

It is an object of the present invention to provide the method for providing multiple medical tests to the user, further comprising selecting, using a graphical user interface coupled to the computing device, at least one of the one or more possible alterations.

It is an object of the present invention to provide the method for providing multiple medical tests to the user, further comprising receiving information from a remote server, the remote server being coupled to one or more secondary computing devices.

It is an object of the present invention to provide the method for providing multiple medical tests to the user, wherein the one or more displays are selected from the group consisting of a 2-dimensional television screen, a 3-dimensional television screen, a 2-dimensional computer display, a 3-dimensional computer display, a projector, and a virtual reality apparatus.

### BRIEF DESCRIPTION OF THE DRAWINGS

FIG. 1 shows a system for providing multiple medical tests to a user, according to an embodiment of the present invention.

FIGS. 2-3 show a method for providing multiple medical tests to a user, according to an embodiment of the present invention.

FIG. 4 shows a graph of an exponential growth curve resulting from a Cardio-pulmonary Tolerance Test, according to an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Described herein is an approach to human medical testing and sensing technologies that incorporates multiple medical testing and sensing technologies incorporating hardware and software in order to perform a plurality of medical tests. According to an embodiment, the multiple medical testing and sensing technologies may include a PulseOx, an ECG, a spirometry sensor, a skin conductance sensor, an infrared camera, an EMG, an EEG, a facial expression recognition sensor, a capnometer, a pneumograph, a photoplethismogram, a rheoencephalogram, a hemoencephalogram, an ultrasound sensor, an ultrasound transducer, and/or any other suitable or relevant testing and/or testing technologies while maintaining the spirit of the present invention.

According to an embodiment, one or more of the medical testing and/or sensing technologies may be controlled by a single centralized computing device. According to an embodiment, the centralized computing device may include a processor and a memory. According to an embodiment, the memory may include software configured to control the one or more medical testing and/or sensing technologies connected to and/or incorporated into the centralized hub and/or configured to perform diagnostic testing using the one or more medical testing and/or sensing technologies.

The use of multiple, separate diagnostic machines and devices increases the costs to medical providers. In order to offset these costs, the price to patients and insurance companies is then increased. By incorporating multiple medical devices and testing equipment into a singular device, the present invention is an improvement upon the existing technologies and also saves medical professionals' and patients' money.

According to an embodiment, a Clinician Timeline Editor is provided with the present invention for a clinician to create a custom treatment by dragging and dropping a wide range of Negative and Positive Interventions so as to tailor treatment to a patient. The clinician may use already pre-determined diagnosis specific timelines and treatment protocols and/or custom diagnosis specific timelines and treatment protocols.

According to an embodiment, the present invention incorporates biofeedback that adds multiple layers to traditional biofeedback by adding the concept of ambience. Such an approach is herein referred to as “Ambiofeedback”. The ambience in question pertains to the high level of photorealism present in a display component of the present invention, as well as the wide array of environments and procedural uses of interactive pacers present in the Ambiofeedback approach. Pacers are used in biofeedback to guide patients to performing various tasks such as, e.g., breathing, movement of one or more limbs, etc.

According to an embodiment, the ambience is also determined by a wide array of possible interventions possible with the use of the present invention such as, e.g., therapeutic interventions, educational interventions, and other relevant forms of interventions. According to an embodiment, apparatus of the present invention may have access to a library of diagnosis related treatment timelines and protocols to choose from.

The Ambiofeedback is made possible by a new type of biofeedback unit. In traditional biofeedback, the biofeedback unit is primarily a sensor signal translator and the clinician puts out positive interventions (relaxation, guided imageries, breathing techniques, etc.) or negative interventions (stress inducers, etc.) and measures the responses from the patients. In Ambiofeedback, the biofeedback unit provides sophisticated graphics and data manipulation, as well as the use of a wide library of protocols and timelines, interventions, environments, pacers, and educational material, all integrated in the biofeedback treatment.

Ambiofeedback is a diagnostic, therapy and research tool and can profoundly influence the outcome of biofeedback treatment as well as the way the clinicians adapt their interventions.

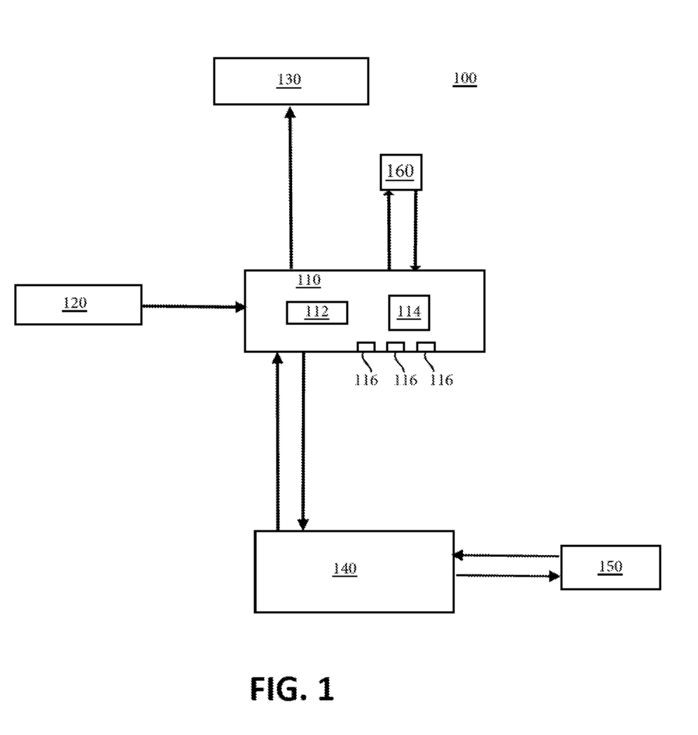

Referring now to FIG. 1, a system 100 for providing medical testing to a user is illustratively depicted, in accordance with an embodiment of the present invention.

According to an embodiment, the system 100 includes a centralized computing device 110 (hereinafter the “Unit”), one or more sensors 120, and a display 130. According to an embodiment, the system 100 further includes a connection 130 to a remote server 140 (such as, e.g., a cloud server), which is connected to one or more secondary Units 150. According to an embodiment, the remote server 150 assists the Unit 110 in treatments, graphic displays, program updates, and/or in collecting treatment data for comparative analysis. Educational material (charts, graphics, data, explanations, etc.) may also be sent to the Unit 110 through the server 140 to be used by Clinicians and other users. According to an embodiment, the Unit 110 is coupled to the one or more sensors 120 and the display 130 by a wired and/or wireless connection. According to an embodiment, one or more of the one or more sensors 120 may be incorporated into the Unit 110. According to an embodiment, the Unit 110 may be a component of the one or more sensors 120 and/or the display 130. According to an embodiment, the Unit 110 may be a multi-function apparatus which serves as a multi-testing unit and/or an ambiofeedback unit.

According to an embodiment, the Unit 110 includes several ports 116 for the connecting of one or more sensors 120. According to an embodiment, the one or more sensors may include a PulseOx, an ECG, a spirometry sensor, a skin conductance sensor, an infrared camera, an EMG, an EEG, a facial expression recognition sensor, a capnometer, a pneumograph, a photoplethismogram, a rheoencephalogram, a hemoencephalogram, an ultrasound, and/or any other suitable or relevant sensor while maintaining the spirit of the present invention. According to an embodiment, the sensors 120 are configured to send data to the Unit 110 for analysis. According to an embodiment, one or more of the sensors 120 are incorporated into the Unit 110.

According to an embodiment, the Unit 110 includes a processor 112 and a memory 114. The Unit 110 is configured to analyze data captured by the one or more sensors 120. According to an embodiment, the memory includes instructions that, when executed by the processor, cause the Unit 110 to send a signal to the display 130. According to an embodiment, the signal causes the display 130 to project relevant data and/or analyzed results from the one or more sensors 120. According to an embodiment, the signal causes the display 130 to project a relevant visual representation for the Ambiofeedback procedure which may include, e.g., a photorealistic display of an environment, one or more dashboard overlays, interactive graphics, and/or any other relevant visual stimuli. According to an embodiment, the Unit 110 is configured to be used by a Clinician. However, it is noted that the Unit 110 may be configured to be used by other individuals.

According to an embodiment, each of the one or more sensors 120 is configured to record data results. According to an embodiment, these data results are sent to the Unit 110 and rendered back to one or more Clinicians using the display 130 and/or a graphical user interface 160. According to an embodiment, data from other Units 150 is gathered, either at one of the Units 110, 150 or at the server 140 for data analysis and to generate comparative statistics to aid Clinicians in their treatments and in their diagnostics and testing interpretations.

According to an embodiment, the system 100 further includes a graphical user interface 160 to control the Unit 110 and/or interact with the interactive graphics. The graphical user interface may be coupled to the Unit 110, one or more of the sensors 120, and/or the display 130. According to an embodiment, the graphical user interface 160 may be a component of the Unit 110, one or more of the sensors 120, and/or the display 130.

According to an embodiment, the display 130 may include a television screen (2-dimensional and/or 3-dimensional, a computer display (2-dimensional and/or 3-dimensional), a virtual reality apparatus, an augmented reality apparatus, a projector (2-dimensional and/or 3-dimensional), and/or any other suitable display device that may be used while maintaining the spirit of the present invention. According to an embodiment, the system 100 may include multiple displays 130.

According to an embodiment, specific diagnostics and testing using one or more of the sensors 120 (e.g., ECG, EEG, spirometric sensor, EMG, etc.) is performed independently of any treatment being performed by the system 100. However, according to an embodiment, specific diagnostics and testing using one or more of the sensors 120 may be performed in conjunction with any treatments being performed by the system 100.

According to an embodiment, treatment may include patient education, psychoeducation, and/or psychotherapeutic applications and may by displayed using the display 130. According to an embodiment, treatment may include biofeedback treatments with Ambiofeedback technology (e.g., the Unit 110 and the display 130), producing an ambient environment in conjunction with the biofeedback treatment.

According to an embodiment, the Unit 110 is configured to perform a Cardio-pulmonary Tolerance Test (CPTT). The CPTT includes measuring the behavior of the heart and lungs of the subject, thus picking up the efficacy and strength of the heart-lung interaction and monitoring the overall health of the heart lung dyad. From this, a specific value, herein called the Cardio-pulmonary Tolerance Index, is able to be determined.

The CPTT utilizes the biofeedback capability of the Unit 110. According to an embodiment, the patient is connected to a PulseOx sensor and the pulse rate is measured. The patient is asked to follow a Pacer rhythm on the screen 130 and asked to breath in and out based on the rhythm of the pacer. According to an embodiment, the pacer is set to start at 10 breaths a minute for a determined period of time (1-2 minutes). After that time, the pacer is set to reduce the rhythm to 9 breaths a minute for an approximately equal amount of time. After that, the pacer is once again reduced to 8 breaths a minute, then 7 breaths a minutes and then 6 breaths a minute, each with approximately equal increments of time, using an integer-based reduction called the Breathing Step Reduction Phase.

After a certain amount of reduction, during each breathing cycle, the heart rate of the patient begins to oscillate between a maximum heart rate value and a minimum heart rate value, based on inhaling and exhaling. This is called the Respiratory Sinus Arrhythmia (RSA).

The CPTT measures the Power of the Oscillation of the RSA (P). The formula for the Power of Oscillation is defined as

$$P = \frac{{A}^{2}}{F} ,$$

wherein A is the Amplitude of the Oscillation (the difference between the maximum heart rate value and the minimum heart rate value) and F is the Frequency of the pacer or the breathing rate of the patient.

During the Breathing Step Reduction Phase, the Unit picks up two values: the Amplitude of the first Oscillation that is measured when it appears, called the Minimum Amplitude (or “A min”; and its corresponding breathing rate called the Minimum Frequency or the Breathing where this first oscillation started (or “F min”). From those two values, the Minimum Power of Oscillation (or “P min”) is obtained.

From there starts the Breathing Progressive Reduction Phase. During this phase, the patient continues to follow the pacer, which, at this phase, reduces progressively rather than in incremental steps. This allows the Amplitude of the Oscillation to increase. At one point, when the patient cannot keep up breathing according the now very reduced frequency of the pacer, the Oscillation breaks down. Just prior to the Oscillation breaking down, two more values are recorded: the Maximum Amplitude (or “A max”) and its corresponding breathing rate, called the Maximum Frequency (or “F max”). From those two values, the Maximum Power of Oscillation (or “P max”) is obtained.

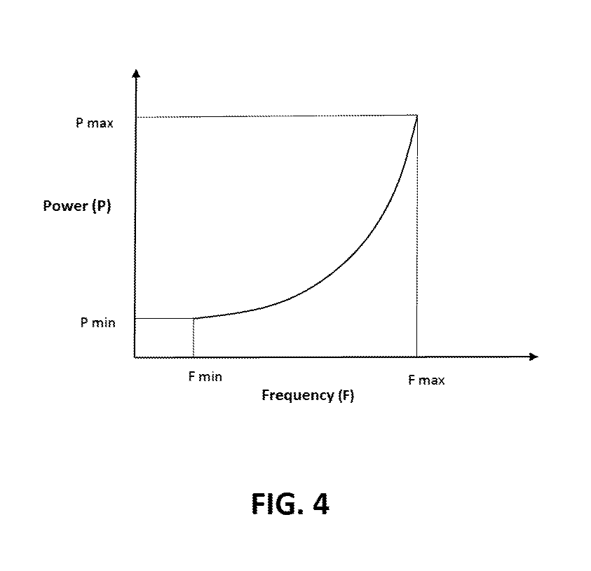

The test is now completed and an Exponential Growth Curve (as shown in FIG. 4) can be plotted between P min and the P max and the corresponding Frequencies.

The exponential curve obeys the Exponential Growth/Decay formula:

x(t)=x₀×(1+R)^{t}

The t (or time factor) is, in the case of the CPTT, replaced by the Frequency. X(t) is the P max, and X₀ is the P min. R is the rate of growth of the curve.

The rate of growth of that curve, R, is the Cardio-pulmonary Tolerance Index which is determined by the following formula:

$$R = \sqrt[Fmax]{\frac{Pmax}{Pmin}} - 1$$

The Cardio-pulmonary Tolerance Index is an indicator of the cardiac and pulmonary health of the individual. It can be used to monitor treatment progress on multiple treatments such as, e.g., pulmonary or cardiac rehab, pulmonary treatments biofeedback treatments, etc. It can also be potentially used a predicting indicator for pathology and treatment outcomes and can be used in research.

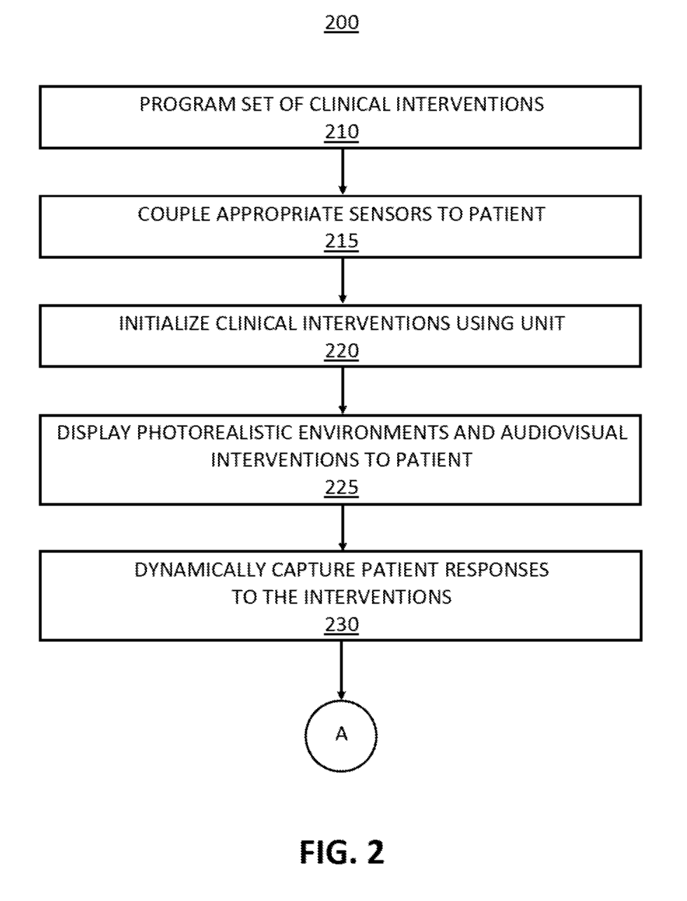

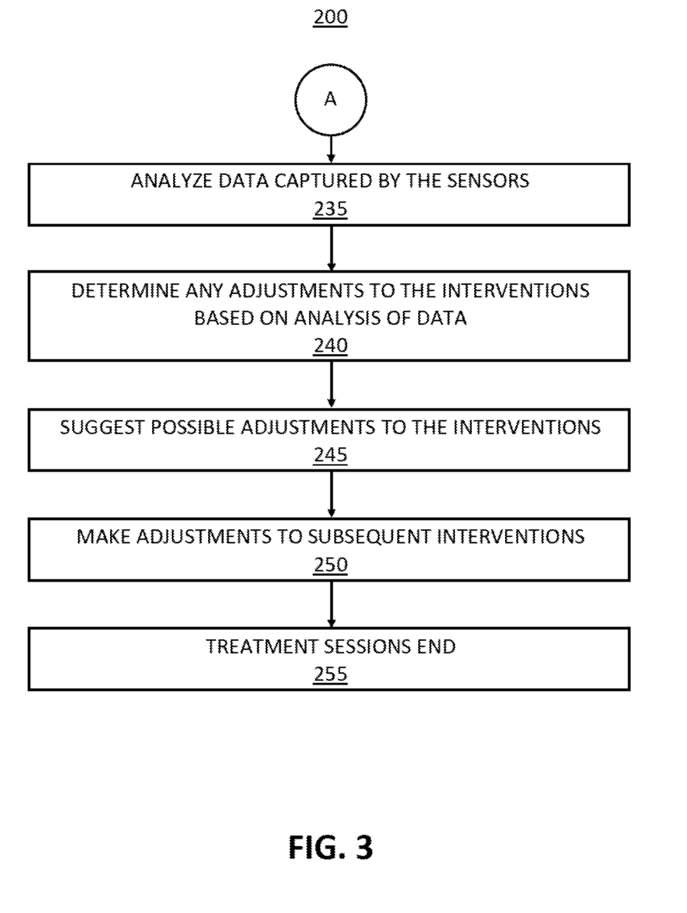

Referring now to FIGS. 2-3, a method 200 for providing medical testing to a user is illustratively provided, in accordance with an embodiment of the present invention.

At step 210, a clinician or other suitable professional programs a set of clinical interventions to be run by the Unit 110 over a session treatment timeline.

At step 215, the clinician or other suitable professional couples one or more appropriate sensors 120 to the patient. According to an embodiment, the one or more sensors 120 may include a PulseOx, an ECG, a spirometry sensor, a skin conductance sensor, an infrared camera, an EMG, an EEG, a facial expression recognition sensor, a capnometer, a pneumograph, a photoplethismogram, a rheoencephalogram, a hemoencephalogram, an ultrasound sensor, an ultrasound transducer, and/or any other suitable or relevant sensor while maintaining the spirit of the present invention.

At step 220, the Unit 110 initializes the clinical interventions. The clinical interventions include displaying (at step 225), on a dynamic physiologic display 130, photorealistic environments and audiovisual interventions that may include one of more pacers, which are visual guides that guide patients to performing various tasks such as, e.g., breathing, movement of one or more limbs, etc. For example, a pacer may have a rhythm which may guide the breathing rate of a patient. According to an embodiment, the display 130 may also display graphic testing results, such as, for example, ECG, EEG, and Spirometry results, among other suitable results.

The interventions used for a particular patient may be in line with specific biofeedback treatment protocols that are designed to diagnose and/or treat specific ailments (e.g., asthma, COPD, anxiety, PTSD, etc.) and are adaptable to demographic patient information (e.g., age, weight, etc.) and/or co-morbid conditions. According to an embodiment, the interventions may be psychotherapeutic interventions (e.g., cognitive, supportive, etc.) It is noted, however, that other types of interventions may also be administered while maintaining the spirit of the present invention.

At step 230, the Unit 110 dynamically captures the patient's responses to the interventions by capturing and analyzing data from the one or more sensors 120 coupled to the patient. At step 235, the Unit 110 analyzes the data, based on prior patient data and data from other patients. According to an embodiment, the Unit 110 may receive data from one or more servers 140 coupled to the Unit 110. The one or more servers 140 may be coupled to one or more secondary Units 150.

Based on the analysis of the patient data, the Unit 110, at step 240, determines any adjustments to the interventions in the timeline of interventions. According to an embodiment, the Unit 110, at step 245, provides suggested adjustments to the intervention protocol to the clinician or other suitable professional and also provides live feedback. According to an embodiment, the Unit 110 displays the suggestions to the Clinician or other suitable professional using the display 130 and/or the graphical user interface 160.

At step 250, the Unit 110 alters subsequent interventions in the timeline of interventions based on the determined adjustments. According to an embodiment, the treatment protocols are adaptable in that the treatment protocols measure patient performance and adapt and adjust one or more treatments/interventions accordingly in a step fashion. This enables full interactivity between the patient and the treatment.

According to an embodiment, the Unit 110 determines the adjustments automatically. According to an embodiment, the Unit 110 suggests one or more adjustments and/or new interventions to the clinician or other suitable professional using the display 130 and/or another suitable graphical user interface 160 and the clinician approves of any changes to the intervention timeline. The adjustments may be based, in whole or in part, on a library of treatment protocols which may be housed within the Unit 110 and/or accessible on the server 140.

At step 255, the intervention treatments are complete and the session treatment timeline of interventions ends.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for providing multiple medical tests to a user, comprising:
a computing device, the computing device including:
a memory configured to store user data one or more clinical interventions; and
a processor configured to run the one or more clinical interventions;
one or more sensors coupled to the computing device, each of the one or more sensors configured to capture data from the user and send the data to the computing device;
one or more displays coupled to the computing device, the one or more displays configured to display the one or more clinical interventions; and
a remote server coupled to the computing device.

2. The system as recited in claim 1, wherein at least one of the one or more sensors are wirelessly coupled to the computing device.

3. The system as recited in claim 1, wherein the computing device further includes:
one or more adapters configured to receive one or more inputs from the one or more sensors.

4. The system as recited in claim 1, wherein the one or more sensors are selected from the group consisting of:
a PulseOx;
an ECG;
a spirometry sensor;
a skin conductance sensor;
an infrared camera;
an EMG;
an EEG;
a facial expression recognition sensor;
a capnometer;
a pneumograph;
a photoplethismogram;
a rheoencephalogram;
a hemoencephalogram;
an ultrasound sensor; and
an ultrasound transducer.

5. The system as recited in claim 1, wherein the computing device is configured to cause the display to project a photorealistic display of an environment to coincide with one or more clinical interventions.

6. The system as recited in claim 1, wherein the computing device is configured to analyze data captured by the one or more sensors in order to determine one or more possible alterations to a regimen of clinical interventions in a timeline of interventions.

7. The system as recited in claim 6, wherein the computing device is configured to display, to a second user, the one or more possible alterations.

8. The system as recited in claim 1, wherein the one or more sensors are incorporated into the computing device.

9. The system as recited in claim 1, wherein the remote server is coupled to one or more secondary computing devices.

10. The system as recited in claim 1, wherein the one or more displays are selected from the group consisting of:
a 2-dimensional television screen;
a 3-dimensional television screen;
a 2-dimensional computer display;
a 3-dimensional computer display;
a projector;
a virtual reality apparatus; and
an augmented reality apparatus.

11. A method for providing multiple medical tests to a user, comprising:
programming, on a computing device, a series of clinical interventions to be performed by a user, the computing device including:
a memory configured to store user data one or more clinical interventions; and
a processor configured to run the one or more clinical interventions;
coupling one or more sensors to the user,
wherein the one or more sensors are coupled to the computing device;
displaying to the user, on one or more displays, one or more clinical interventions from the series of clinical interventions;
capturing data from the user, using the one or more sensors;
sending the data to the computing device; and
analyzing the data, using the computing device.

12. The method as recited in claim 11, wherein at least one of the one or more sensors are wirelessly coupled to the computing device.

13. The method as recited in claim 11, wherein the computing device further includes:
one or more adapters configured to receive one or more inputs from the one or more sensors.

14. The method as recited in claim 11, wherein the one or more sensors are selected from the group consisting of:
a PulseOx;
an ECG;
a spirometry sensor;
a skin conductance sensor;
an infrared camera;
an EMG;
an EEG;
a facial expression recognition sensor;
a capnometer;
a pneumograph;
a photoplethismogram;
a rheoencephalogram;
a hemoencephalogram;
an ultrasound sensor; and
an ultrasound transducer.

15. The method as recited in claim 11, further comprising sending a signal from the computing device to the one or more displays to cause the one or more displays to project a photorealistic display of an environment to coincide with one or more clinical interventions.

16. The method as recited in claim 11, wherein the analyzing the data further includes determining one or more possible alterations to series of clinical interventions based on the data.

17. The method as recited in claim 16, further comprising displaying, to a second user, the one or more possible alterations.

18. The system as recited in claim 16, further comprising selecting, using a graphical user interface coupled to the computing device, at least one of the one or more possible alterations.

19. The method as recited in claim 11, further comprising receiving information from a remote server, the remote server being coupled to one or more secondary computing devices.

20. The method as recited in claim 11, wherein the one or more displays are selected from the group consisting of:
a 2-dimensional television screen;
a 3-dimensional television screen;
a 2-dimensional computer display;
a 3-dimensional computer display;
a projector;
a virtual reality apparatus; and
an augmented reality apparatus.
